(19) United States
(12) Patent Application Publication
(10) Pub. No.: US 2008/0255657 A1
(43) Pub. Date: Oct. 16, 2008

(54) STENT WITH UNCONNECTED STENT SEGMENTS

(75) Inventors: Daniel Gregorich, St. Louis Park, MN (US); Michael Meyer, Richfield, MN (US); Soo-Young Yoon, Maple Grove, MN (US); Richard Tooley, Crystal, MN (US)

Correspondence Address: JONES DAY, 222 EAST 41ST ST, NEW YORK, NY, 10017, US

(73) Assignee: Boston Scientific Scimed, Inc., Maple Grove, MN (US)

(21) Appl. No.: 12/098,622

(22) Filed: Apr. 7, 2008

Related U.S. Application Data
(60) Provisional application No. 60/910,698, filed on Apr. 9, 2007.

(51) Int. Cl. A61F 2/82

(52) U.S. Cl. 623/1.16; 623/1.42; 623/1.43

(57) ABSTRACT

This invention generally relates to stents, such as intravascular stents. More particularly, the invention is directed to stents comprising expandable stent segments, such as unconnected expandable stent segments. The invention is also directed to methods for making such stents.

### CROSS-REFERENCE TO RELATED APPLICATIONS

This application claims the benefit of U.S. Provisional Application No. 60/910,698, filed on Apr. 9, 2007, the entire contents of which are incorporated herein by reference.

### FIELD OF THE INVENTION

This invention generally relates to stents, such as intravascular stents. More particularly, the invention is directed to stents comprising expandable stent segments, such as unconnected expandable stent segments. The invention is also directed to methods for making such stents.

### BACKGROUND OF THE INVENTION

Stents are used to treat a variety of medical conditions. In blood vessels, they have been used to treat, e.g., stenoses and aneurysms. They are also used to treat or correct conditions in body lumens other than blood vessels, such as the ureter, urethra, duodenum, and bile duct. Drug-coated stents are known, for localized delivery of therapeutic agents to a body lumen. See, e.g., U.S. Pat. No. 6,099,562 to Ding et al.

Certain stents comprise a plurality of expandable stent segments interconnected by connectors. Such stents can be manufactured by a variety of methods. For example, they can be manufactured by starting with a hollow cylinder made of a given material. Struts and connectors are formed by removing material from certain parts of the cylinder.

It has been noted that the performance and characteristics of stents can be improved. For example, one aspect of the performance of a stent in a medical application is deliverability, i.e. the ability of the stent to be delivered through sometimes tortuous body lumens. To some degree, deliverability is affected by the number of connectors included in a stent. Reducing the number of connectors tends to make the stent more flexible or bendable, leading to increased deliverability of the stent.

Also, another characteristic of stents is uniformity or non-uniformity of design parameters along the length of the stent. For example, a stent may have a consistent geometric design along its entire length, or the stent may have a geometric design that varies along the length. In part because of current methods of manufacture, it is difficult to make a stent having non-uniform design parameters along its length, as opposed to uniform design parameters along its length.

It is therefore an object of the present invention to provide stents having improved deliverability and more flexibility in the uniformity or non-uniformity of design parameters along the length of the stent.

### SUMMARY OF THE INVENTION

The present invention seeks to address these objectives by providing a stent comprising individual expandable stent segments not connected to each other whose design parameters can be readily varied along the length of the stent. Also, since the stent segments are unconnected, deliverability of the stent is increased.

In one embodiment, an intravascular stent for implantation in a blood vessel is disclosed comprising a first expandable stent segment comprised of a first material and a second expandable stent segment comprised of a second material that is different from the first material, wherein the first stent segment and the second stent segment may or may not be connected to each other. The first material may have a different radiopacity than the second material. For instance, the second material may have a greater radiopacity than the first material. In some embodiment, the first material may comprise platinum. The first and second materials may each comprise a weight percentage of platinum, and the second material may have a higher weight percentage of platinum than the first material. Also, the second stent segment may be disposed at one end of the stent. In another aspect of this embodiment, the stent may further comprise a third expandable stent segment that is not connected to the first and second stent segments. The third stent segment may comprise the second material and be disposed at the other end of the stent. The third stent segment may comprise a third material that is different from the first and second materials. The stent segments may be configured to be expanded using a balloon.

In another embodiment, an intravascular stent for implantation in a blood vessel is disclosed comprising at least three expandable stent segments that are not connected to each other, wherein the first stent segment comprises a first material and the second and third stent segments comprise a second material that is more radiopaque than the first material, and wherein the first stent segment is disposed between the second and third stent segments and the second and third stent segments are each disposed at an end of the stent.

In yet another embodiment, an intravascular stent for implantation in a blood vessel is disclosed comprising a first expandable stent segment having a first composition comprising a first therapeutic agent disposed thereon, and a second expandable stent segment having a second composition comprising a second therapeutic agent disposed thereon, wherein the first and second stent segments may or may not be connected to each other and wherein the first and second therapeutic agents are different. The first therapeutic agent may comprise an anti-proliferative agent. The first therapeutic agent may comprise an anti-thrombotic agent. The first therapeutic agent may comprise an anti-proliferative agent, and the second therapeutic agent may comprise an anti-thrombotic agent. In a variation, the stent further comprises a third expandable stent segment that is not connected to the first and second stent segments. The first stent segment may be disposed between the second and third stent segments and the third stent segment may comprise the second composition comprising the second therapeutic agent disposed thereon. The second and third stent segments may each be disposed at an end of the stent. The first therapeutic agent may comprise paclitaxel. The first therapeutic agent may comprise rapamycin, and if so the second therapeutic agent may comprise paclitaxel. The first therapeutic agent may comprise an anti-inflammatory agent. The stent may be configured to be expanded using a balloon.

In yet another embodiment, a stent for implantation in a blood vessel is disclosed comprising a first expandable stent segment having a first composition comprising a first amount of a therapeutic agent disposed thereon, and a second expandable stent segment having a second composition comprising a second amount of the therapeutic agent disposed thereon, wherein the second amount is different from the first amount, and wherein the first and second stent segments may or may not be connected to each other. The first amount may be less than the second amount. The first composition may comprise a first polymer, and the second composition may comprise a second polymer. In a variation, the stent further comprises a third expandable stent segment that may or may not be connected to the first and second stent segments. A third composition comprising a third amount of the therapeutic agent may be disposed thereon. The third amount may be different from the first amount and the second amount. The first stent segment may be disposed between the second and third stent segments. The stent may be configured to be expanded using a balloon.

In yet another embodiment, a stent for implantation in a blood vessel is disclosed comprising at least three stent segments that may or may not be connected to each other, wherein the first stent segment is disposed between the second and third stent segments and the second and third stent segments are each disposed at an end of the stent, and wherein the first stent segment comprises a first amount of a composition comprising a therapeutic agent disposed thereon and the second and third stent segments each comprise a second amount of the composition comprising the therapeutic agent disposed thereon in which the first amount is less than the second amount.

In yet another embodiment, a stent for implantation in a blood vessel is disclosed comprising a first expandable stent segment having an outer surface having a first surface area, and a second expandable stent segment having an outer surface having a second surface area, wherein the first and second stent segments may or may not be connected to each other, and wherein the first surface area and second surface area are different. The first expandable stent segment may comprise struts having a first undulation length and the second expandable stent segment may comprises struts having a second undulation length. The first undulation length may be less than the second undulation length.

In yet another embodiment, a stent for implantation in a blood vessel is disclosed comprising a first expandable stent segment having a first strut thickness, and a second expandable stent segment having a second strut thickness, wherein the first and second stent segment may or may not be connected to each other. The second strut wall thickness may be greater than the first strut wall thickness.

In yet another embodiment a stent for implantation in a blood vessel is disclosed comprising at least three stent segments that may or may not be connected to each other and are disposed about a longitudinal axis, wherein the first stent segment is disposed between the second and third stent segments and the second and third stent segments are each disposed at an end of the stent, and wherein the first stent segment comprises a plurality of struts having a first strut thickness, and wherein the second and third stent segments each comprises a plurality of struts having a second strut thickness, and wherein the second strut thickness is greater than the first strut thickness.

In another embodiment, a stent for implantation in a blood vessel comprises a first expandable stent segment having a plurality of struts. These struts include at least a first strut that has an abluminal surface, a luminal surface, an upstream surface, and a downstream surface. The stent also includes a second expandable stent segment having a plurality of struts. These struts of the second stent segment include at least a second strut that has an abluminal surface, a luminal surface, an upstream surface, and a downstream surface. A first coating is disposed on at least one of the abluminal, luminal, upstream or downstream surfaces of the first strut. A second coating is disposed on at least one of the abluminal, luminal, upstream or downstream surfaces of the second strut. The first and second coatings are disposed on different surfaces or on a different combination of surfaces of the first and second strut. Also, the first and second stent segments are not connected to each other.

### BRIEF DESCRIPTION OF THE DRAWINGS

Preferred features of the present invention are disclosed in the accompanying drawings, wherein similar reference characters denote similar elements throughout the several views, and wherein:

FIG. 1 shows an exemplary stent with unconnected stent segments made of different materials;

FIG. 2 shows an exemplary stent with unconnected stent segments having different therapeutic agents;

FIG. 3 shows another exemplary stent with unconnected stent segments having different therapeutic agents;

FIG. 4A shows an exemplary stent with unconnected stent segments having different amounts of a coating composition;

FIG. 4B shows another exemplary stent with unconnected stent segments having different amounts of a coating composition;

FIG. 5 shows examples of stent segments having different surface areas;

FIG. 6A shows examples of stent segments having certain dimensions;

FIG. 6B is a cross-sectional view of the strut of FIG. 6A at plane X1-X1;

FIG. 7A shows a cross-sectional view of a stent with unconnected segments in which a coating is disposed on a different combination of surfaces of the struts in each segment;

FIG. 7B shows a cross-sectional view of a another stent with unconnected segments in which a coating is disposed on different surfaces of the struts in each segment;

FIG. 8A shows end views of stent segments having different strut thicknesses;

FIG. 8B shows a side view of a stent comprising the stent segments of FIG. 7A; and

FIGS. 9A-9D show an exemplary delivery of a stent into a body lumen.

### DETAILED DESCRIPTION OF THE INVENTION

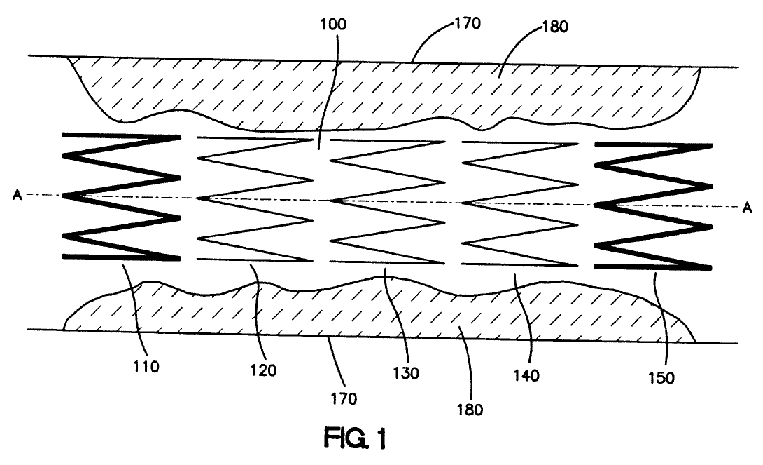

In one embodiment, the unconnected stent segments comprising a stent may be made of different materials. In the exemplary embodiment depicted in FIG. 1, stent 100 is shown as delivered within body lumen 170, and adjacent to obstruction 180. The portion of body lumen 170 in the vicinity of delivered stent 100 can be called the “stented area.” Stent 100 comprises stent segments such as stent segments 110, 120, 130, 140, and 150 that comprise different materials and are not connected to each other. In alternative embodiments some or all the segments can be connected. Stent 100 has a longitudinal axis A-A. In a preferred embodiment, at least one of stent segments 110, 120, 130, 140, and 150 comprises a different material from at least one other of stent segments 110, 120, 130, 140, and 150. For example, stent segment 110 may comprise platinum or a platinum alloy, and stent segment 120 may comprise stainless steel. Stent segment 130 may comprise a material having a certain weight percentage of platinum, and stent segment 140 may comprise a material having a different (e.g. higher) weight percentage of platinum. Other materials that stent segments 110-150, or any of the other stent segments described herein, may comprise include, without limitation, metallic, ceramic, or polymeric material, or a combination thereof. Suitable metal materials include, but are not limited to, gold, platinum, stainless steel, titanium, tantalum, iridium, molybdenum, niobium, palladium or chromium. Suitable polymers include, but are not limited to, ethylene-vinylacetate copolymers, such as, polyethylene-co-vinyl acetate; polymethacrylates, such as, poly(n-butyl methacrylate); styrene-isobutylene copolymers, such as, poly(styrene-b-, isobutylene-b-styrene); and polylactic acids, such as, polylactic-glycolic acid.

In some variations, each stent segment may comprise a different material from every other stent segment. In other variations, at least some of the stent segments may comprise the same material. Also, these stent segments of different materials can include a coating comprising a polymer and/or therapeutic agent such as those discussed below.

The materials comprising stent segments 110-150 may be chosen to have different properties, such as radiopacity, tensile strength, malleability, density, porosity, hardness, heat conductivity, electrical conductivity, magnetic susceptibility, coefficient of thermal expansion, length change on expansion, heat capacity, melting point, hoop strength, radial resistive force, flexibility, stiffness, elastic recoil or shape memory properties. For example, a stent segment that is located at the end of the stent (i.e., an end stent segment), such as stent segment 110, may be chosen to have a greater radiopacity than a stent segment, such as stent segment 120, that is not an end stent segment. This may permit the ends of the stent to be readily identified using X-ray photography or fluoroscopy. As another example, stent segments 120, 130, and 140 may be chosen to have high flexibility to improve delivery of the stent through tortuous passages, while stent segments 110 and 150 may be chosen to have high hoop strength in order to prevent stent collapse. Other variations and combinations are expressly contemplated, and will be appreciated by those of skill in the art.

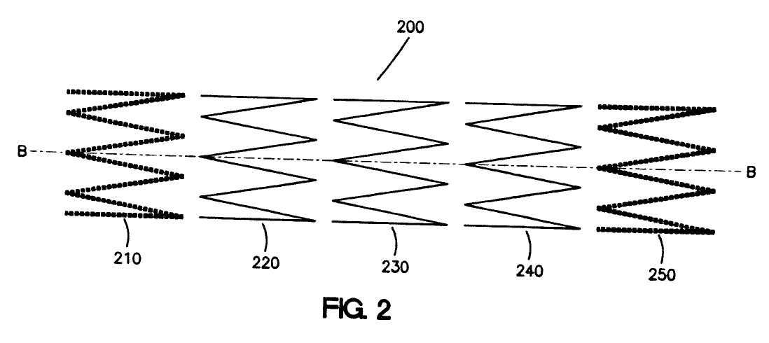

In another embodiment, the stent segments comprise different therapeutic agents. For instance, in the exemplary embodiment depicted in FIG. 2, stent 200 comprises stent segments 210, 220, 230, 240, and 250 and has a longitudinal axis B-B. In this embodiment, stent segments 210 and 220 have different therapeutic agents disposed upon them, and are not connected to each other. In other embodiments, some or all of the segments may be connected to each other. For therapeutic agents with relatively high diffusivity, this may allow delivery of each of the different therapeutic agents to essentially the entire stented area, in effect delivering a “drug cocktail” thereto. For therapeutic agents with relatively lower diffusivity, this may allow delivery of each particular therapeutic agent to a particular part of the stented area, thereby treating only tissue in the vicinity of a stent segment comprising that particular therapeutic agent. The fact that the stent segments are not connected to each other facilitates the manufacture of such a stent, because individual stent segments can be separately coated with the required therapeutic agent.

For example, stent segment 210 may be coated with an anti-thrombotic drug, and stent segment 220 may be coated with an anti-proliferative drug. Stent segment 210 may be coated with rapamyacin or a rapamyacin derivative. Stent segment 220 may be coated with paclitaxel. Stent segment 210 may be coated with an anti-inflammatory. Various other therapeutic agents including but not limited to those described herein may be placed on the stent segments. A more comprehensive (though not exhaustive) listing of therapeutic agents with which the stent segments of stent 200 may be coated appears infra.

In some variations, each stent segment 210-250 may be coated with a different therapeutic agent, such as a drug. In other embodiments, at least some of the stent segments may be coated with the same therapeutic agent. In some embodiments, such as that of FIG. 2, the end stent segments (e.g. stent segments 210 and 250) are coated with one therapeutic agent, and the stent segments that are not end stent segments (e.g. stent segments 220, 230, 240) are coated with a different therapeutic agent. For example, an end stent segment can be coated with a therapeutic agent that is intended to be delivered to the blood stream, while a stent segment that is not an end stent segment can be coated with a therapeutic agent that is intended to be delivered to the obstruction by contact with the stent segment.

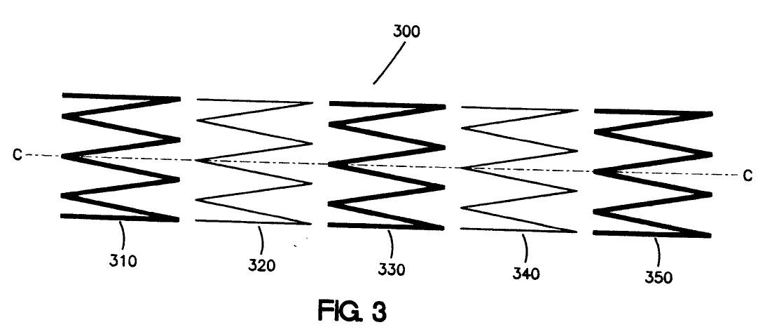

In the exemplary stent of FIG. 3, stent 300 is comprised of stent segments 310, 320, 330, 340, 350, and has a longitudinal axis C-C. In this embodiment, stent segments 310-350 are coated with two different therapeutic agents in an alternating pattern. For example, stent segment 310 may be coated with a first therapeutic agent, stent segment 320 may be coated with a second therapeutic agent, and stent segment 330 may be coated with the first therapeutic agent. An alternating pattern may repeat along the entire length of stent 300 or, alternatively, along only a portion of the length of stent 300. In a preferred embodiment, the first therapeutic agent is sirolimus and the second therapeutic agent is paclitaxel. An alternating pattern may facilitate the delivery of a “drug cocktail” to the stented area. For example, it may be that a drug cocktail comprising two drugs must be delivered to the stented area, but it may not be possible to coat a given stent segment with both drugs. This may be because one drug is not compatible with another. As another example, it may be because one drug is optimally delivered on a stent segment having a certain physical configuration and the other drug is optimally delivered on a stent segment having another physical configuration. In such cases, the drug cocktail can be delivered by coating the drugs on alternating stent segments.

In another variation, the stent segments are coated with three or more different therapeutic agents in a repeating pattern. For example, stent segment 310 may be coated with a first therapeutic agent, stent segment 320 may be coated with a second therapeutic agent, stent segment 330 may be coated with a third therapeutic agent, stent segment 340 may be coated with the first therapeutic agent, stent segment 350 may be coated with the second therapeutic agent, and a sixth stent segment (not shown) adjacent to stent segment 350 may be coated with the third therapeutic agent.

Many other variations are possible. For example, if the numerals 1, 2, 3, . . . represent a first therapeutic agent, a second therapeutic agent, and a third therapeutic agent, a series of stent segments might be coated with therapeutic agents in the pattern 1-2-2-1-1-2-2-1, or in the pattern 1-2-3-3-3-1-2-3-3-3, or in any other pattern. In another embodiment, stent segments having different therapeutic agents disposed thereon are not arranged in a repeating pattern. Such an embodiment may be advantageous for an application in which a stent must have some certain number of stent segments coated with a given therapeutic agent, but for which the relative placement of such stent segments along the stent is unimportant. In general, stent segments having different therapeutic agents disposed thereon can be arranged in any order, depending on the needs of the application.

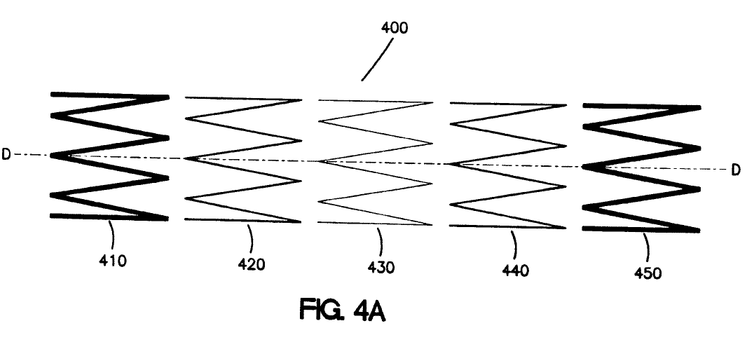

Also, the therapeutic agent disposed on the stent segments can be contained in a coating. The coating can contain in addition to the therapeutic agent a polymer, such as those discussed below. In the exemplary embodiment depicted in FIG. 4A, stent 400 comprises stent segments 410, 420, 430, 440, 450, and has a longitudinal axis D-D. In this embodiment, stent segments 410 and 420 are coated with different amounts of a therapeutic agent, and are not connected to each other. In other embodiments, some or all of the segments may be connected to each other. For example, stent segment 410 may have a relatively greater amount of a composition containing a first therapeutic agent, and stent segment 420 may have a smaller amount of the coating composition, such that there may be a greater amount of the first therapeutic agent on stent segment 410 than on stent segment 420. Alternatively, a greater amount of the therapeutic agent on stent segment 410 may be achieved by disposing a coating composition having a higher concentration (e.g. the weight percentage or atomic percentage) of the therapeutic agent on stent segment 410, and disposing a composition that comprises a lower concentration of the therapeutic agent on stent segment 420.

In the embodiment shown in FIG. 4A, stent segments 410 and 450, which are end stent segments, have the relatively greatest amount of therapeutic agent disposed thereon, and stent segment 430, the middle stent segment, has the relatively least amount of therapeutic agent. Stent segments 420 and 440 have an intermediate amount of therapeutic agent disposed thereon.

In another embodiment, shown in FIG. 4B, stent 405 comprises stent segments 415, 425, 435, 445, 455, with the middle stent segment, stent segment 435, having the most therapeutic agent. In this embodiment, the amount of coating is reduced toward the end stent segments 415 and 425, which have the least therapeutic agent.

Many other variations are possible. For example, if the numerals 1, 2, 3, . . . represent a first amount, a second amount, and a third amount of therapeutic agent, a series of stent segments might be coated with different amounts of therapeutic agent in the pattern 1-2-2-1-1-2-2-1, or in the pattern 1-2-3-3-3-1-2-3-3-3, or in any other pattern. This may allow delivery of specific amounts of therapeutic agent to specific tissue areas based on, for example, the specific configuration of a particular obstruction within a lumen.

Patterns that load more drug near the ends of the stent may be advantageous in achieving uniform delivery, considering that the vessel beyond an end of the stent may act as a “sink,” absorbing more of the drug, whereas the segments in the middle are surrounded by adjacent drug-eluting segments. Such configurations may be represented by patterns such as 2-1-1-1-1-2, 2-2-1-1-1-2-2, 3-2-1-1-1-2-3, or 5-4-3-2-1-2-3-4-5, where higher numbers indicate relatively larger amounts of drug or therapeutic agent.

In another variation, the therapeutic agent amount of the unconnected stent segments comprising a stent may vary randomly, subject to the constraints that no single stent segment has a therapeutic agent amount of greater than M1 grams or less than M2 grams, and the total therapeutic agent amount of all the stent segments in the stent taken together is no greater than M3 grams and no less than M4 grams, where M1, M2, M3, and M4 are variables that can be assigned particular numeric values. This variation may have the advantage that less precise, less costly drug application methods may be used in making the stent while still delivering the correct overall amount of therapeutic agent to the stented area.

Varying the amount of the coating along the stent can achieve a more uniform distribution of the therapeutic agent to tissues along the stent, or, alternatively, can achieve a deliberately non-uniform distribution of the therapeutic agent. For example, if the therapeutic agent is soluble or suspendable in blood, sorting the stent segments such that the stent segment with the smallest amount of therapeutic agent is at the downstream end of the stent may achieve a more uniform distribution of drug to the tissue surrounding the stent, since the amounts of therapeutic agent contributed by the stent segments will tend to be additive in the downstream direction.

In another embodiment, each of the unconnected stent segments comprising a stent may be coated with a composition comprising a therapeutic agent having a different release rate. The release rate may be affected by, for example, the matrix or polymers in which the therapeutic agent is disposed. In a variation, the release rates may vary from unconnected stent segment to unconnected stent segment within a stent according to a pattern. In another variation, the release rates may vary from unconnected stent segment to unconnected stent segment within a stent randomly.

In another embodiment, each of the unconnected stent segments comprising a stent may be coated with a therapeutic agent (or a composition comprising a therapeutic agent) having a different duration of release. In a variation, the release durations may vary from unconnected stent segment to stent segment within a stent according to a pattern. In another variation, the release durations may vary from unconnected stent segment to stent segment within a stent randomly.

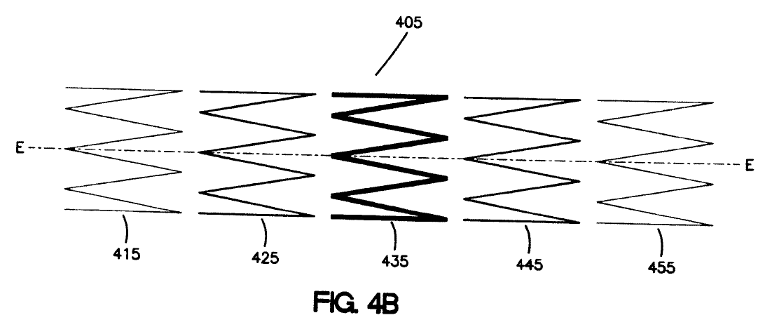

FIG. 5 depicts examples of stent segments that may vary in total undulation length due to variations in strut geometry or size. For example, stent segment 510 may have a greater undulation length than stent segment 520 because stent segment 510 has a greater number of peaks and valleys than stent segment 520. Consequently, stent segment 510 also may have a greater total surface area than stent segment 520.

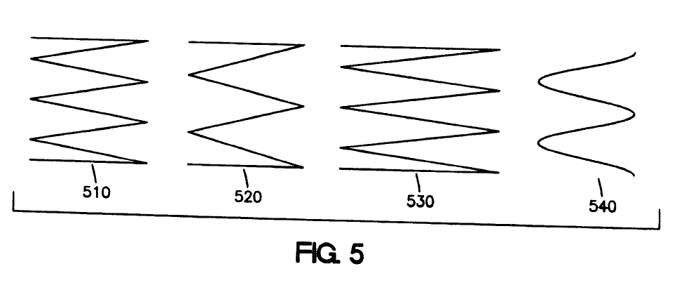

FIG. 6A points out the dimensions of stent segments that can affect the total undulation length and total surface area of a stent segment, including the length L of the stent segment, the height H of the stent segment, the length L′ of a strut, the angle θ between struts, and the radius of curvature R of a curved strut. FIG. 6B which is a cross-sectional view of a strut of segment 540 across line XI-XI, depicts two other stent segment dimensions, in particular the width W and thickness T of a typical strut. Depending on the values of these dimensions, stent segments such as stent segments 510, 520, 530, 540 depicted in FIG. 5 may differ in undulation length and surface area.

In one embodiment, the stent comprises unconnected stent segments that vary in total undulation length, or vary in the total surface area of an outer surface, or in both total undulation length and total surface area. In one embodiment, a stent comprises end stent segments having a relatively large number of struts per segment, such as stent segment 510, as shown in FIG. 5, and stent segments that are not end stent segments having a relatively lesser number of struts per stent segment, such as stent segment 520, as shown in FIG. 5. This has the effect of increasing the surface area of the end stent segments relative to the other stent segments. When a coating comprising a therapeutic agent is applied to the stent segments, because of their increased surface area, the end stent segments, will have a relatively larger amount of therapeutic agent disposed thereon for delivery to body tissue.

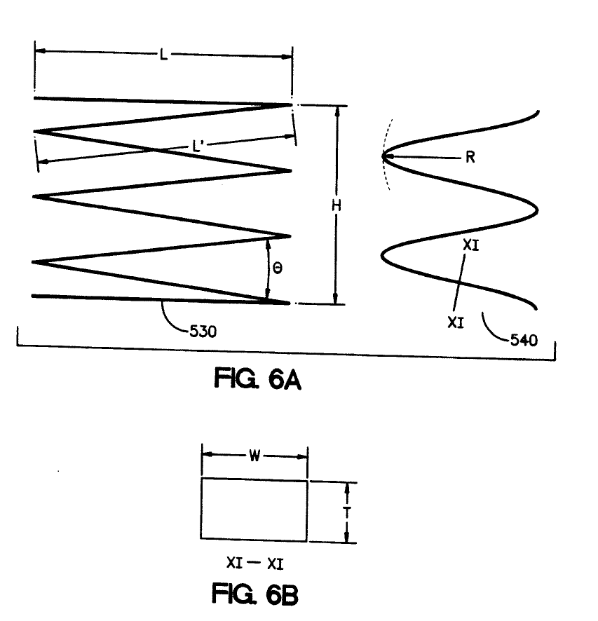

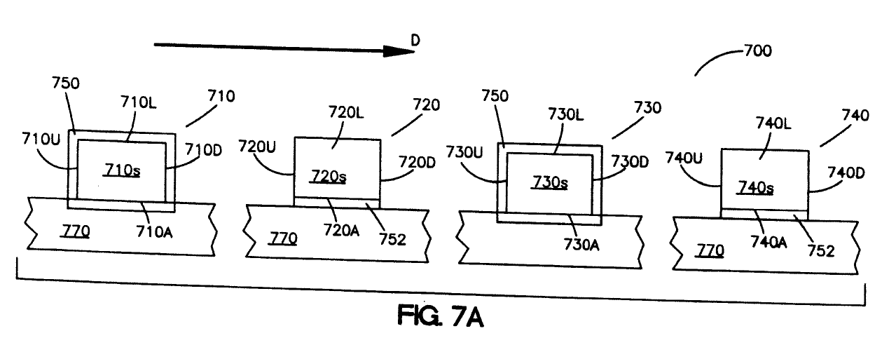

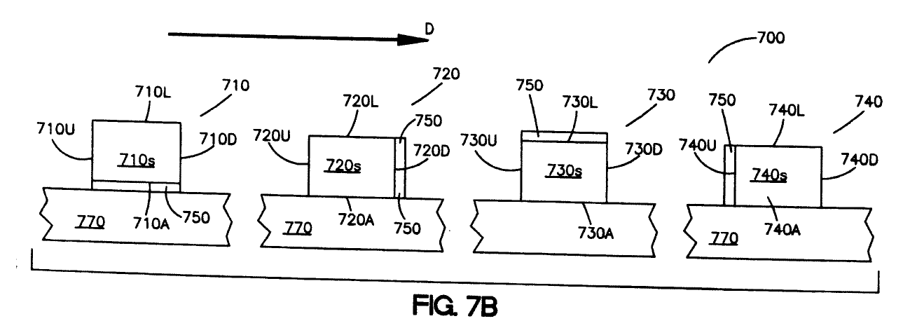

In some embodiments, the stent segments that make up the stent can each have struts in which different surfaces or a different combination of surfaces of the struts are coated with a coating composition. FIG. 7A shows a cross-sectional view of portions of a stent 700 that is implanted in a lumen having a lumen wall 770. Blood flow or body fluid flow is indicated by direction D. The stent 700 is made up of four stent segments, 710, 720, 730 and 740. Each stent segment has at least one strut, e.g. stent strut 710s is part of segment 710, stent strut 720s is part of stent segment 720, stent strut 730s is part of segment 730, and stent strut 740s is part of stent segment 740. Each strut of each stent segment has an abluminal surface 710A, 720A, 730A and 740A, which faces the lumen wall 770 and a luminal surface 710L, 720L, 730L, and 740L, which faces the center of the lumen. Also, each strut has an upstream surface 710U, 720U, 730U and 740U, which faces against direction D and a downstream surface 710D, 720D, 730D and 740D, which faces in direction D.

In this embodiment, the struts of at least two stent segments, 710 and 720, have coatings disposed on a different combination of their respective strut surfaces. Specifically, a first coating 750 is disposed on all four surfaces, 710A, 710L, 710U and 710D of strut 710s of segment 710 and a second coating 752 is only disposed on the abluminal surface 720A of strut 720s of segment 720. Thus, in this embodiment, the first and second coatings are disposed on a different combination of surfaces of the two struts, i.e., four surfaces of strut 710s are coated while one surface of strut 720s is coated. Although the coatings in this embodiment are different, they can be the same. Similarly, a first coating 750 is disposed on all four surfaces, 730A, 730L, 730U and 730D of strut 730s of segment 730 and a second coating 752 is only disposed on the abluminal surface 740A of strut 740s of segment 740.

FIG. 7B shows another embodiment where different surfaces of the struts of two or more stent segments are coated. A first coating 750 is disposed on the abluminal surface 710A of strut 710s of segment 710 and the coating 750 is disposed on the downstream surface 720D of strut 720s of segment 720. Therefore, different surfaces of the struts of segment 710 and 720 are coated. Also in this embodiment, the coating 750 is disposed on the luminal surface 730L of strut 730s of segment 730 and the coating 750 is disposed on the upstream surface 740U of strut 740s of segment 740. Although the coating in this embodiment is disposed on four different strut surfaces of each of the four segments, a coating is considered to be disposed on different strut surfaces as long as those surfaces are different for at least 2 stent segments. Also, different coating compositions can be disposed on the stent segments.

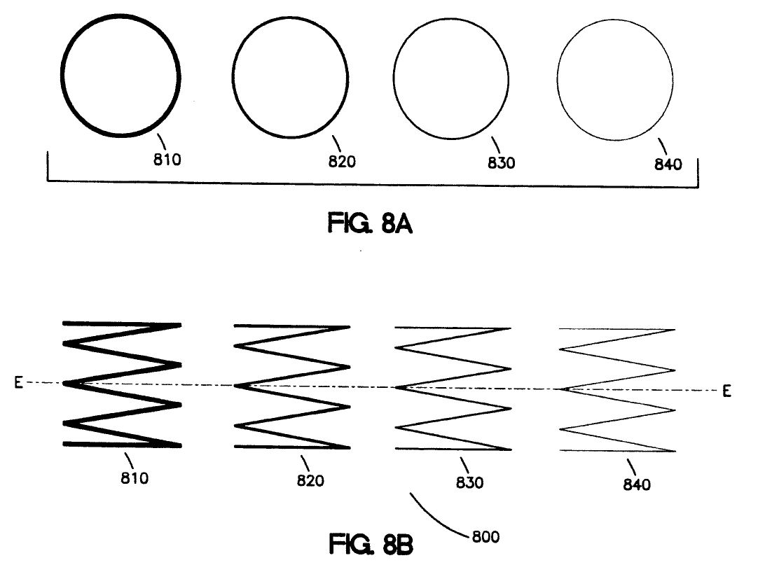

In another embodiment, the inventive stent comprises unconnected stent segments disposed about a longitudinal axis, wherein the thickness of each stent segment is different. For example, FIG. 8A depicts end views of each of the stent segments comprising a stent. That is, in FIG. 8A, the stent segments 810, 820, 830, 840 are arranged in the order in which they appear in the stent, but each is turned to show an end view, thereby displaying the wall thickness of each. The assembled stent 800 is shown in side view in FIG. 8B. Stent 800 has a longitudinal axis E-E. The overall wall thickness of the stent segments tapers, i.e., stent segment 810 has the greatest wall thickness and stent segment 840 has the least wall thickness. If stent segment 810 is at the proximal end and stent segment 840 is at the distal end, such a taper may be advantageous in pushing the stent through a narrow lesion.

In a variation of this embodiment, the wall thickness tapers from the center to the ends (i.e. the central stent segment has the greatest wall thickness and the end stent segments have the least wall thickness). This configuration makes the stent as flexible as possible at the ends, and stiffest in the center. It also tends to maximize laminar flow through the stent. In another variation, wall thickness tapers from the ends toward the center (i.e. the end segments have the greatest wall thickness and the central stent has the least wall thickness in order to position thinner segments centrally). This may provide for more flexible stent segments near the center in order to facilitate delivery through tortuous passages.

Although the exemplary stents described herein and depicted in the above figures generally comprise 4 or 5 stent segments, the inventive stent may comprise other numbers of stent segments, for example 2, or 3, or more than 5 stent segments. In general, a stent segment may have a length less than that of the area to be stented, so that a stent will comprise at least two stent segments. Fewer stent segments may be needed when treating a relatively short section of tissue, and more stent segments may be needed to treat a longer section of tissue. The number of stent segments required will also depend on the length of each individual stent segment.

Even though, in the exemplary stents described herein no stent segment is rigidly connected to any other stent segment, in other variations at least some of the stent segments may be connected to other stent segments. This allows combining the advantages of unconnected stent segments (described supra) with advantages of conventional, connected stent segments, such as greater rigidity. Stent segments may be connected with at least one connecting member.

The exemplary stents described herein may have a tubular or cylindrical-like configuration. However, these stents need not be completely cylindrical. For instance, the cross-section of the stent, or of any of the stent segments of which it is comprised, can be substantially triangular, rectangular, oval-shaped, polygonal, or variable-shaped. The cross-section of a stent can be varied to depend on the shape of a body lumen.

Stent segments are depicted in FIGS. 1-8B as comprising struts arranged in a particular pattern, such as a substantially zig-zag pattern. However, other structures, such as chevron-shaped struts, serpentine struts, mesh structures, and others, may be employed to create other patterns, depending on desired properties such as expandability, flexibility, rigidity, high hoop strength, etc.

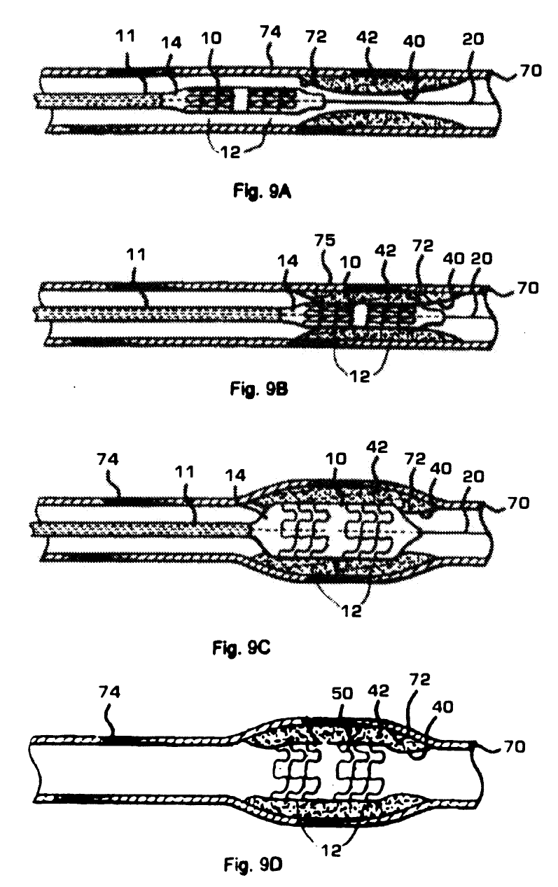

The stent segments of which each stent depicted in the various figures is comprised are shown as disposed along a longitudinal axis, for example the axis A-A of FIG. 1. For example, the stent segments may be disposed along the longitudinal axis of a catheter or balloon, or along the longitudinal axis of a body lumen, as depicted in FIGS. 9A-9D. The stent segments may also be disposed along a variable axis not associated with a specific physical structure.

The stent segments depicted in each of FIGS. 1-8B is comprised may be configured to be expanded using a balloon, as shown in FIGS. 9A-9D.

FIGS. 9A-9D show an exemplary delivery of a stent 10 into a body lumen. Stent 10 comprises unconnected stent segments 12. Stent 10 may first be mounted onto an inflatable balloon 14, or other mechanical delivery system, on the distal end of a delivery catheter 11. Stent 10 may be crimped or collapsed in substantially congruent dimensions to balloon 14. Guidewire 20 may be coaxially disposed in the body lumen prior to the introduction of the stent 10. Stent 10 and catheter 11 may then be introduced into a patient's body. Stent 10 and catheter 11 may be advanced over guidewire 20, at least to the area of obstruction 42. It may be preferable to advance the stent 10 until it is substantially centered in the area of obstruction 42.

Alternatively, the unconnected stent segments comprising stent 10 may be delivered individually (one at a time) or in groups, by balloon or other delivery mechanism.

When stent 10 is inserted into a desired location within a patient, balloon 14 may be inflated, which may thereby expand stent 10. At least one strut element 50 of stent 10 may thereby be brought into contact with at least a portion of the surface 40 of the obstruction 42 and/or the inner wall 72 of a vessel 70. Vessel 70 may be expanded slightly by the expansion of stent 10 to provide volume for the expanded lumen. As a result, interference of blood flow by stent 10 may be minimized, in addition to preventing unwarranted movement of stent 10 once the expansion is complete.

The coating suitable for use with the stents can include a therapeutic agent and/or polymer.

The term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, paclitaxel (as well as its derivatives, analogs or paclitaxel bound to proteins, e.g. Abraxane™) 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g. IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus, everolimus, tacrolimus, pimecrolimus and zotarolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

Polymers useful in the coating composition should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers.

When the polymer is being applied to a part of the medical device, such as a stent, which undergoes mechanical challenges, e.g. expansion and contraction, the polymers are preferably selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. The polymer is selected to allow the coating to better adhere to the surface of the strut when the stent is subjected to forces or stress. Furthermore, although the coating can be formed by using a single type of polymer, various combinations of polymers can be employed.

Generally, when a hydrophilic therapeutic agent is used then a hydrophilic polymer having a greater affinity for the therapeutic agent than another material that is less hydrophilic is preferred. When a hydrophobic therapeutic agent is used then a hydrophobic polymer having a greater affinity for the therapeutic agent is preferred. However, in some embodiments, a hydrophilic therapeutic agent can be used with a hydrophobic polymer and a hydrophobic therapeutic agent can be used with a hydrophilic polymer.

Examples of suitable hydrophobic polymers or monomers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by Ra SiO 4-a/2, where R is a monovalent substituted or unsubstituted hydrocarbon radical and the value of a is 1 or 2; and naturally occurring hydrophobic polymers such as rubber.

Examples of suitable hydrophilic polymers or monomers include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl (meth)acrylate and 2-hydroxypropyl (meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Other suitable polymers include without limitation: polyurethanes, silicones (e.g., polysiloxanes and substituted polysiloxanes), and polyesters, styrene-isobutylene-copolymers. Other polymers which can be used include ones that can be dissolved and cured or polymerized on the medical device or polymers having relatively low melting points that can be blended with biologically active materials. Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluoropolymers, fluorosilicones, polyethylene glycol, polysaccharides, phospholipids, and combinations of the foregoing.

The coating composition comprising the therapeutic agent and/or polymer can be formed using a solvent. Solvents that may be used to prepare coating compositions include ones which can dissolve or suspend the polymer and/or therapeutic agent in solution. Examples of suitable solvents include, but are not limited to, tetrahydrofuran, methylethylketone, chloroform, toluene, acetone, isooctane, 1,1,1, trichloroethane, dichloromethane, isopropanol, IPA, and mixture thereof.

The coating composition comprising the therapeutic agent and/or polymer can be applied to the device by any method. Examples of suitable methods include, but are not limited to, spraying such as by conventional nozzle or ultrasonic nozzle, dipping, rolling, electrostatic deposition, and a batch process such as air suspension, pan coating or ultrasonic mist spraying. Also, more than one coating method can be used.

Coating compositions can be applied selectively to certain surfaces of certain stent segments and not to other surfaces. For example, a certain coating composition can be applied to the abluminal surfaces of one stent segment, whereas a different coating composition is applied to the luminal surfaces of another stent segment. Such selective coating of segments can be more easily accomplished if the segments are not connected to each other. Other techniques, such as masking, may also be used.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed:

1. An intravascular stent for implantation in a blood vessel comprising:
a first expandable stent segment having a first composition comprising a first therapeutic agent disposed thereon; and
a second expandable stent segment having a second composition comprising a second therapeutic agent disposed thereon;
wherein the first and second stent segments are not connected to each other and wherein the first and second therapeutic agents are different.

2. The stent of claim 1, wherein the first therapeutic agent comprises an anti-proliferative agent.

3. The stent of claim 1, wherein the first therapeutic agent comprises an anti-thrombotic agent.

4. The stent of claim 1, wherein the first therapeutic agent comprises an anti-inflammatory agent.

5. The stent of claim 1, further comprising a third expandable stent segment that is not connected to the first and second stent segments and wherein the first stent segment is disposed between the second and third stent segments and the third stent segment comprises the second composition comprising the second therapeutic agent disposed thereon.

6. The stent of claim 5, wherein the second and third stent segments are each disposed at an end of the stent.

7. The stent of claim 1, wherein the first therapeutic agent comprises paclitaxel.

8. The stent of claim 1, wherein the first therapeutic agent comprises rapamycin.

9. An intravascular stent for implantation in a blood vessel comprising:
a first expandable stent segment having a first composition comprising a first therapeutic agent disposed thereon; and
a second expandable stent segment having a second composition comprising a second therapeutic agent disposed thereon;
wherein the first and second therapeutic agents are different.

10. The stent of claim 9, wherein the first therapeutic agent comprises an anti-proliferative agent.

11. The stent of claim 9, wherein the first therapeutic agent comprises an anti-thrombotic agent.

12. The stent of claim 9, wherein the first therapeutic agent comprises an anti-inflammatory agent.

13. The stent of claim 9, further comprising a third expandable stent segment and wherein the first stent segment is disposed between the second and third stent segments and the third stent segment comprises the second composition comprising the second therapeutic agent disposed thereon.

14. The stent of claim 9, wherein the first therapeutic agent comprises paclitaxel.

15. The stent of claim 9, wherein the first therapeutic agent comprises rapamyacin.

16. An intravascular stent for implantation in a blood vessel comprising at least three expandable stent segments that are not connected to each other,
wherein the first stent segment is disposed between the second and third stent segments and the second and third stent segments are each disposed at an end of the stent, and
wherein the first stent segment comprises a first composition comprising an anti-thrombotic agent disposed thereon and the second and third stent segments each comprise a second composition comprising an antiproliferative agent disposed thereon.

17. An intravascular stent for implantation in a blood vessel comprising at least three expandable stent segments that are not connected to each other,
wherein the first stent segment is disposed between the second and third stent segments and the second and third stent segments are each disposed at an end of the stent, and
wherein the first stent segment comprises a first composition comprising an anti-inflammatory agent disposed thereon and the second and third stent segments each comprise a second composition comprising an antiproliferative agent disposed thereon.

18. An intravascular stent for implantation in a blood vessel comprising at least three expandable stent segments that are not connected to each other,
wherein the first stent segment is disposed between the second and third stent segments and the second and third stent segments are each disposed at an end of the stent, and
wherein the first stent segment comprises a first composition comprising an anti-inflammatory agent disposed thereon and the second and third stent segments each comprise a second composition comprising an anti-thrombotic agent disposed thereon.

19. A stent for implantation in a blood vessel comprising:
a first expandable stent segment having a first composition comprising a first amount of a therapeutic agent disposed thereon; and
a second expandable stent segment having a second composition comprising a second amount of the therapeutic agent disposed thereon wherein the second amount is different from the first amount, and
wherein the first and second stent segments are not connected to each other.

20. The stent of claim 19, wherein the first amount is less than the second amount.

21. The stent of claim 19, wherein the first composition comprises a first polymer, and the second composition comprises a second polymer.

22. The stent of claim 19, further comprising a third expandable stent segment that is not connected to the first and second stent segments, and wherein a third composition comprising a third amount of the therapeutic agent is disposed on the third segment, and wherein the third amount is different from the first amount and the second amount.

23. A stent for implantation in a blood vessel comprising at least three stent segments that are not connected to each other,
wherein the first stent segment is disposed between the second and third stent segments and the second and third stent segments are each disposed at an end of the stent, and
wherein the first stent segment comprises a first amount of a composition comprising a therapeutic agent disposed thereon and the second and third stent segments each comprise a second amount of the composition comprising the therapeutic agent disposed thereon in which the first amount is less than the second amount.

24. A stent for implantation in a blood vessel comprising:
a first expandable stent segment having an outer surface having a first surface area, and
a second expandable stent segment having an outer surface having a second surface area,
wherein the first and second stent segments are not connected to each other, and
wherein the first surface area and second surface area are different.

25. The stent of claim 24 wherein the first expandable stent segment comprises struts having a first undulation length and the second expandable stent segment comprises struts having a second undulation length, and
wherein the first undulation length is less than the second undulation length.

26. A stent for implantation in a blood vessel comprising:
a first expandable stent segment having a first strut thickness; and
a second expandable stent segment having a second strut thickness;
wherein the first and second stent segment are not connected to each other, and wherein the first and second strut thicknesses are different.

27. The stent of claim 26, wherein the second strut thickness is greater than the first strut thickness.

28. A stent for implantation in a blood vessel comprising at least three stent segments that are not connected to each other,
wherein the first stent segment is disposed between the second and third stent segments and the second and third stent segments are each disposed at an end of the stent, and
wherein the first stent segment comprises a plurality of struts having a first strut thickness, and
wherein the second and third stent segments each comprises a plurality of struts having a second strut thickness,
and wherein the second strut thickness is greater than the first strut thickness.

29. A stent for implantation in a blood vessel comprising:
a first expandable stent segment having a plurality of struts including at least a first strut having an abluminal surface, a luminal surface, an upstream surface, and a downstream surface; and
a second expandable stent segment having a plurality of struts including at least a second strut having an abluminal surface, a luminal surface, an upstream surface, and a downstream surface;
a first coating disposed on at least one of the abluminal, luminal, upstream or downstream surfaces of the first strut,
a second coating disposed on at least one of the abluminal, luminal, upstream or downstream surfaces of the second strut,
wherein the first and second coatings are disposed on different surfaces or on a different combination of surfaces of the first and second strut, and
wherein the first and second stent segments are not connected to each other.

30. The stent of claim 29, wherein at least the abluminal surface of the first strut is coated with the first coating.

31. The stent of claim 29, wherein at least the luminal surface of the first strut is coated with the first coating.

32. The stent of claim 29, wherein at least the upstream surface of the first strut is coated with the first coating.

33. The stent of claim 29, wherein at least the downstream surface of the first strut is coated with the first coating.

34. The stent of claim 29, wherein the abluminal, luminal, upstream and downstream surfaces of the first strut are coated with the first coating.
